(19) United States
(12) Patent Application Publication
(10) Pub. No.: US 2006/0034792 A1
(43) Pub. Date: Feb. 16, 2006

(54) Cosmetic composition based on a cationic surfactant, an amino silicone or vinyl silicone, a fatty alcohol and a propellant

(75) Inventors: Pascale Lazzeri, Levallois-Perret (FR); Jean-Michel Sturla, Boulogne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/192,031

(22) Filed: Jul. 29, 2005

Related U.S. Application Data
(60) Provisional application No. 60/609,829, filed on Sep. 15, 2004.

(30) Foreign Application Priority Data
Aug. 11, 2004 (FR) 04 08824

(51) Int. Cl. A61K 8/89; A61K 8/49

(52) U.S. Cl. 424/70.12; 424/70.28; 424/47

(57) ABSTRACT

The present patent application relates to a cosmetic composition, especially a hair composition, wherein it comprises, in a cosmetically acceptable medium, at least one cationic surfactant, at least one silicone chosen from amino silicones and vinyl silicones, at least one fatty alcohol, and at least one propellant.

This application claims priority to U.S. provisional application 60/609,829 filed Sep. 15, 2004, and to French patent application 04 08824 filed Aug. 11, 2004, both incorporated herein by reference.

The present invention relates to novel cosmetic compositions, especially hair compositions, comprising at least one cationic surfactant, at least one silicone chosen from amino silicones and vinyl silicones, at least one fatty alcohol, and at least one propellant. The invention is also directed towards the use of these compositions in cosmetics, and in particular the use of these compositions as hair conditioners.

Standard hair conditioners in cream form allow good cosmetic properties to be obtained on the hair, such as smoothing, disentangling and gloss. However, the user occasionally has the sensation that his hair is heavy and lank, and easily becomes greasy again, most particularly if his hair is fine. In addition, the cream is occasionally difficult to measure out, and the user consumes a lot of product during each application.

The Applicant has just discovered, surprisingly, that foaming compositions comprising at least one cationic surfactant, at least one silicone chosen from quaternized or non-quaternized amino silicones, and vinyl silicones, at least one fatty alcohol, and at least one propellant, allow the drawbacks mentioned above to be overcome.

The Applicant has in particular found that these compositions are rich and creamy, and stay in the hand very well.

The Applicant has also found that it is not necessary to use these compositions in large amounts, and that, during application, they distribute and spread easily from the roots to the ends, and are particularly easy to rinse out.

Finally, the Applicant has found that these compositions give the hair good cosmetic properties, especially gloss, disentangling and smoothing, the hair is light and becomes greasy again less quickly, and the hair can be shaped very easily and durably.

One subject of the present invention is thus a cosmetic composition, especially a hair composition, wherein it comprises, in a cosmetically acceptable medium, at least one cationic surfactant, at least one silicone chosen from quaternized or non-quaternized amino silicones, and vinyl silicones, at least one fatty alcohol, and at least one propellant.

A subject of the invention is also an aerosol device comprising a composition according to the invention.

Another subject of the invention concerns a cosmetic haircare process using the compositions according to the invention.

A subject of the invention is also the use of the compositions according to the invention for the cosmetic treatment of the hair and the scalp.

Another subject of the invention is the use of the composition for washing and/or conditioning keratin materials such as the hair.

Other characteristics, aspects, subjects and advantages of the invention will emerge even more clearly on reading the description and the examples that follow.

The term “cosmetically acceptable medium” means a medium that is compatible with any keratin material, such as the skin, the hair, the nails, the eyelashes, the eyebrows, the lips and any other area of body or facial skin.

The cosmetically acceptable medium preferably comprises of water or of one or more cosmetically acceptable solvents such as alcohols or water-solvent mixtures. These solvents are preferably C₁-C₄ alcohols.

Among these alcohols, mention may be made of ethanol and isopropanol, ethanol being particularly preferred.

Among the cationic surfactants that may be mentioned in particular (non-limiting list) are: salts of optionally polyoxyalkylenated primary, secondary or tertiary fatty amines; quaternary ammonium salts including alkylpyridinium salts, imidazoline ammonium salts, diquaternary ammonium salts, and ammonium salts containing at least one ester function.

Examples of quaternary ammonium salts that may especially be mentioned include:
those of general formula (V) below: 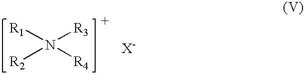
in which the symbols R₁ to R₄, which may be identical or different, represent a linear or branched aliphatic radical containing from 1 to 30 carbon atoms, or an aromatic radical such as aryl or alkylaryl. The aliphatic radicals may comprise hetero atoms such as, especially, oxygen, nitrogen, sulfur and halogens. The aliphatic radicals are chosen, for example, from alkyl, in particular C₁₂-C₂₂ alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals, containing from about 1 to 30 carbon atoms; X⁻ is an anion chosen from the group of halides, phosphates, acetates, lactates, (C₂-C₆) alkyl sulfates and alkyl- or alkylaryl-sulfonates;
quaternary ammonium salts of imidazoline, for instance those of formula (VI) below: 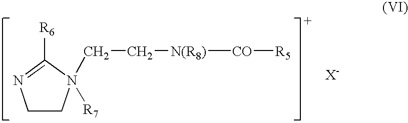
in which R₅ represents an alkenyl or alkyl radical containing from 8 to 30 carbon atoms, for example fatty acid derivatives of tallow or of coconut, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X is an anion chosen from the group of halides, phosphates, acetates, lactates, alkyl sulfates, alkyl sulfonates or alkylaryl sulfonates. R₅ and R₆ preferably denote a mixture of alkenyl or alkyl radicals containing from 12 to 21 carbon atoms, such as, for example, fatty acid derivatives of tallow, R₇ denotes methyl and R₈ denotes hydrogen. Such a product is, for example, Quaternium-27 (CTFA 1997) or Quaternium-83 (CTFA 1997), which are sold under the names “Rewoquat®” W75, W90, W75PG and W75HPG by the company Witco,
diquaternary ammonium salts of formula (VII): 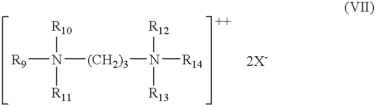
in which R₉ denotes an aliphatic radical containing from about 16 to 30 carbon atoms, R₁₀, R₁₁, R₁₂, R₁₃ and R₁₄, which may be identical or different, are chosen from hydrogen and an alkyl radical containing from 1 to 4 carbon atoms, and X⁻ is an anion chosen from the group of halides, acetates, phosphates, nitrates, ethyl sulfates and methyl sulfates. Such diquaternary ammonium salts in particular comprise propanetallowdiammonium dichloride;
quaternary ammonium salts containing at least one ester function, such as those of formula (VIII) below: 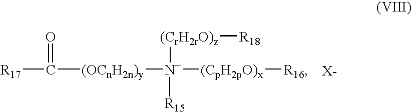
in which:
   R₁₅ is chosen from C₁-C₆ alkyl radicals and C₁-C₆ hydroxyalkyl or dihydroxyalkyl radicals;
   R₁₆ is chosen from:
a radical 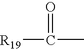
linear or branched, saturated or unsaturated C₁-C₂₂ hydrocarbon-based radicals R₂₀,
a hydrogen atom,
   R₁₇ is chosen from:
a radical 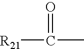
linear or branched, saturated or unsaturated C₁-C₆ hydrocarbon-based radicals R₂₂,
a hydrogen atom,
   R₁₇, R₁₉ and R₂₁, which may be identical or different, are chosen from linear or branched, saturated or unsaturated C₇-C₂, hydrocarbon-based radicals;
   r, n and p, which may be identical or different, are integers ranging from 2 to 6;
   y is an integer ranging from 1 to 10;
   x and z, which may be identical or different, are integers ranging from 0 to 10;
   X⁻ is a simple or complex organic or inorganic anion;
   with the proviso that the sum x+y+z is from 1 to 15, that when x is 0, then R₁₆ denotes R₂₀ and that when z is 0, then R₁₈ denotes R₂₂.

The alkyl radicals R₁₅ may be linear or branched, and more particularly linear.

Preferably, R₁₅ denotes a methyl, ethyl, hydroxyethyl or dihydroxypropyl radical, and more particularly a methyl or ethyl radical.

Advantageously, the sum x+y+z is from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

Advantageously, R₁₇, R₁₉ and R₂₁, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are 0 or 1.

Advantageously, y is equal to 1.

Preferably, r, n and p, which may be identical or different, are equal to 2 or 3 and even more particularly equal to 2.

The anion X⁻ is preferably a halide (chloride, bromide or iodide) or a C₁-C₄ alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function may be used.

The anion X⁻ is even more particularly chloride or methyl sulfate.

Use is made more particularly in the composition according to the invention of the ammonium salts of formula (VIII) in which:
R₁₅ denotes a methyl or ethyl radical,
x and y are equal to 1;
z is equal to 0 or 1;
r, n and p are equal to 2;
R₁₆ is chosen from:
a radical 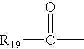
methyl, ethyl or C₁₄-C₂₂ hydrocarbon-based radicals,
   a hydrogen atom;
   R₁₈ is chosen from:
a radical 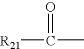
a hydrogen atom;
   R₁₇, R₁₉ and R₂₁, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals.

The hydrocarbon-based radicals are advantageously linear.

Examples of compounds of formula (VIII) that may be mentioned include the salts (especially chloride or methyl sulfate) of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The composition according to the invention may preferably contain a mixture of quaternary ammonium mono-, di- and triester salts with a weight majority of diester salts.

Examples of mixtures of ammonium salts that may be used include the mixture containing 15% to 30% by weight of acyloxyethyl-dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals containing from 14 to 18 carbon atoms and being derived from optionally partially hydrogenated palm oil.

It is also possible to use the ammonium salts containing at least one ester function described in patents U.S. Pat. No. 4,874,554 and U.S. Pat. No. 4,137,180.

Among the quaternary ammonium salts mentioned above that are preferably used are those corresponding to formula (V). Mention may be made firstly of salts (and especially the methosulfates) of dipalmitoylethylhydroxymethylammonium, salts (and especially the chlorides) of tetraalkylammonium, for instance salts (and especially the chlorides) of dialkyldimethylammonium or alkyltrimethylammonium in which the alkyl radical contains from about 12 to 22 carbon atoms, in particular behenyltrimethylammonium, distearyldimethylammonium, cetyltrimethylammonium or benzyldimethylstearylammonium salts (and especially the chlorides), or alternatively, secondly of salts (and especially the chlorides) of palmitylamidopropyltrimethylammonium or salts (and especially the chlorides) of stearamidopropyldimethyl(myristyl acetate)ammonium, and especially the stearamidopropyldimethyl(myristyl acetate)ammonium chloride sold under the name Ceraphyl® 70 by the company Van Dyk.

The cationic surfactants that are even more particularly preferred in the composition of the invention are chosen from quaternary ammonium salts, and in particular from behenyltrimethylammonium chloride, dipalmitoylethylhydroxyethylmethylammonium methosulfate, cetyltrimethylammonium chloride, Quaternium-83, behenylamidopropyl-2,3-dihydroxypropyldimethylammonium chloride and palmitylamidopropyltrimethylammonium chloride.

The cationic surfactant(s) may be present in the composition in a content ranging from 0.01% to 10% by weight, preferably from 0.1% to 5% by weight and more preferably from 0.2% to 4% by weight relative to the total weight of the composition.

According to the invention, the term “amino silicone” denotes any silicone containing at least one primary, secondary or tertiary amine or a quaternary ammonium group. Mention may thus be made of:
(a) the polysiloxanes referred to in the CTFA dictionary as “amodimethicone” and corresponding to the formula: 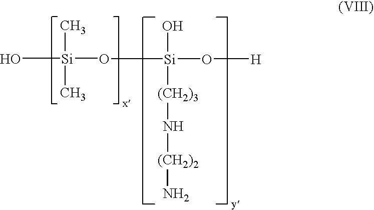
in which x′ and y′ are integers dependent on the molecular weight, generally such that the said number-average molecular weight is between 5000 and 500 000 approximately;
(b) amino silicones corresponding to the formula:
   R′ₐG₃₋ₐ-Si(OSiG₂)ₙ—(OSiG_{b}R′_{2-b})ₘ—O—SiG₃₋ₐ—R+ₐ (IX)
in which:
G is a hydrogen atom or a phenyl, OH or C₁-C₈ alkyl, for example methyl, group,
a denotes the number 0 or an integer from 1 to 3, in particular 0,
b denotes 0 or 1, and in particular 1,
m and n are numbers such that the sum (n+m) can range especially from 1 to 2000 and in particular from 50 to 150, n possibly denoting a number from 0 to 1999 and in particular from 49 to 149 and m possibly denoting a number from 1 to 2000, and in particular from 1 to 10;
R′ is a monovalent radical of formula —C_{q}H_{2q}L in which q is a number from 2 to 8 and L is an optionally quaternized amine group chosen from the groups:
—NR″-Q-N′(R″)2
—N(R″)2
—N⊕(R″)3 A-
—NH⊕(R″)2 A-
—NH2⊕(R″) A-
—N(R″)-Q-N⊕R″H2 A-
—NR″-Q-N⊕(R″)2H A-
—NR″-Q-N⊕(R″)3 A-,
in which R″ can denote hydrogen, phenyl, benzyl or a saturated monovalent hydrocarbon-based radical, for example an alkyl radical containing from 1 to 20 carbon atoms; Q denotes a linear or branched group of formula CᵣH₂ᵣ, r being an integer ranging from 2 to 6 and preferably from 2 to 4; and A⁻ represents a halide ion such as, for example, fluoride, chloride, bromide or iodide.

A product corresponding to this definition is the silicone known as “trimethylsilyl amodimethicone”, corresponding to the formula: 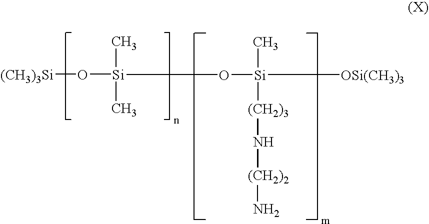
in which n and m have the meanings given above (cf. formula IX).

Such polymers are described, for example, in patent application EP-A-95238.
(c) amino silicones corresponding to the formula: 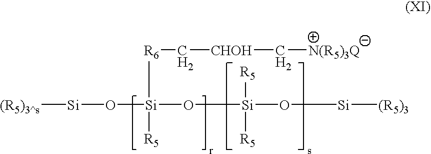
in which:
R₅ represents a monovalent hydrocarbon-based radical containing from 1 to 18 carbon atoms, and in particular a C₁-C₁₈ alkyl or C₂-C₁₈ alkenyl radical, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
Q⁻ is an anion such as a halide ion, in particular chloride, or an organic acid salt (acetate, etc.);
r represents an average statistical value from 2 to 20 and in particular from 2 to 8;
s represents an average statistical value from 20 to 200 and in particular from 20 to 50.

Such amino silicones are described more particularly in patent U.S. Pat. No. 4,185,087.
d) quaternary ammonium silicones of formula: 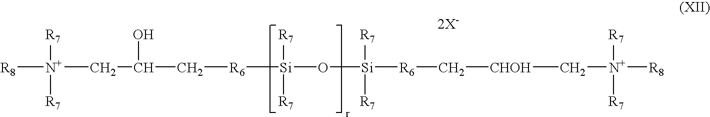
in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide ion, in particular chloride, or an organic acid salt (acetate, etc.);
r represents an average statistical value from 2 to 200 and in particular from 5 to 100.

These silicones are described, for example, in application EP-A-0 530 974.
e) amino silicones of formula (XIII): 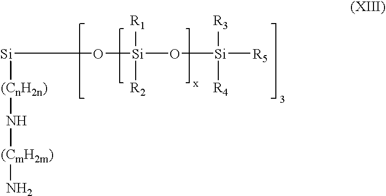
in which:
   R₁, R₂, R₃ and R₄, which may be identical or different, denote a C₁-C₄ alkyl radical or a phenyl group,
   R₅ denotes a C₁-C₄ alkyl radical or a hydroxyl group,
   n is an integer ranging from 1 to 5,
   m is an integer ranging from 1 to 5,
and in which x is chosen such that the amine number is between 0.01 and 1 meq/g.

The silicones that are particularly preferred in accordance with the invention are polysiloxanes containing amine groups, such as amodimethicones or trimethylsilyl amodimethicones (CTFA 4th edition 1997), and even more particularly silicones containing quaternary ammonium groups.

The vinyl silicones may be chosen from silicone copolymers resulting from the addition reaction, in the presence of a catalyst, of at least:
(a) one polysiloxane of formula (I): 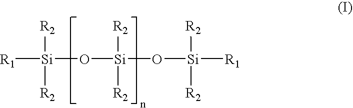
in which:
R1 denotes a group capable of reacting by chain-addition reaction, for instance a hydrogen atom or an ethylenically unsaturated aliphatic group, especially vinyl, allyl or hexenyl,

The groups R₂ of formula (I) may especially represent alkyl, alkenyl, cycloalkyl, aryl, alkylaryl or hydroxyl groups, and may also comprise functional groups such as ethers, amines, carboxyls, hydroxyls, thiols, esters, sulfonates or sulfates.

The alkyl groups contain, for example, from 1 to 20 carbon atoms; the cycloalkyl groups contain, for example, 5 or 6 carbon atoms; the aryl groups are especially phenyl groups; the alkenyl groups especially contain from 2 to 10 carbon atoms; the alkylaryl groups may contain from 7 to 20 carbon atoms.

More particularly, R₂ denotes methyl.

n is an integer such that the polysiloxane of formula (I) preferably has a kinematic viscosity of between 1 and 1×10⁶ mm²/s. n ranges especially from 5 to 5000.
(b) and of at least one silicone compound comprising at least one and not more than two groups capable of reacting with the groups R₁ of the polysiloxane (a),
at least one of the radicals R₁ of the compounds of type (a) or (b) denotes an ethylenically unsaturated aliphatic group.

The compounds of the type (b) are another polysiloxane of the type (a) in which the groups R₁ of the polysiloxane (b) are capable of reacting with the groups R₁ of the polysiloxane (a).

Preferably, the silicone copolymers are especially obtained by addition reaction, in the presence of a hydrosilylation catalyst (for example a platinum catalyst), of at least:
(a) one α,ω-divinyl polydimethylsiloxane, and
(b) of one α,ω-dihydrogeno polydimethylsiloxane.

The kinematic viscosity is measured, for example, at 25° C. according to ASTM standard 445 Appendix C.

The silicone copolymers according to the invention are essentially non-crosslinked.

The synthesis of emulsions based on these silicones is especially described in patent application EP-A-874 017.

Such emulsions are especially sold under the name DC2-1997 Cationic Emulsion by the company Dow Corning. This emulsion comprises an α,\overline{ω}-divinyl dimethicone/α,\overline{ω}-dihydrogeno dimethicone copolymer with a dynamic viscosity of about 15.10⁶ cP, an emulsifier of cationic type such as cetyltrimethylammonium chloride, a stabilizer of hydroxyethylcellulose type, and water.

The vinyl silicone is preferably chosen from polydimethylsiloxane copolymers containing α,ω-divinyl/α,ω-dihydrogeno polydimethylsiloxane groups.

The silicone(s) may be present in the composition in a content of between 0.01% and 6%, preferably between 0.1% and 3% and more preferably between 0.4% and 2% by weight relative to the total weight of the composition.

The fatty alcohol according to the invention may be linear or branched, and saturated or unsaturated, and may contain from 8 to 40 carbon atoms.

The fatty alcohol may be oxyalkylenated or glycerolated.

Preferably, the fatty alcohol is not oxyalkylenated or glycerolated. The fatty alcohol may have the structure R—OH, in which R denotes a linear or branched, saturated or unsaturated radical containing from 8 to 40 and preferably from 8 to 30 carbon atoms; R preferably denotes a C₁₂-C₂₄ alkyl or a C₁₂-C₂₄ alkenyl group. R may be substituted with one or more hydroxyl groups.

Examples that may be mentioned include cetyl alcohol, stearyl alcohol and oleyl alcohol, and mixtures thereof.

According to the invention, the term “oxyalkylenated fatty alcohol” means any pure fatty alcohol having the following structure: 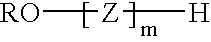
in which:
R denotes a saturated or unsaturated, linear or branched radical containing from 8 to 40 and preferably from 8 to 30 carbon atoms,
Z represents an oxyethylene (i) and/or oxypropylene (ii)1 and (ii)2 radical having the following respective formulae: 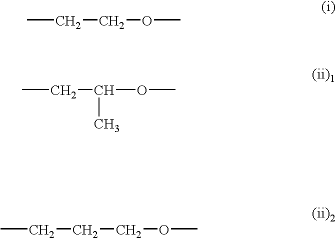
m represents the number of ethylene oxide (i) and/or propylene oxide (ii)1 or (ii)2 groups, ranging from 1 to 250 and preferably from 2 to 100.

The term “glycerolated fatty alcohol” means any pure fatty alcohol having the following structure: 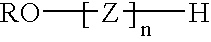
in which:
R denotes a saturated or unsaturated, linear or branched radical containing from 8 to 40 and preferably from 8 to 30 carbon atoms,
Z represents a glycerolated radical (iii) having the following formula: 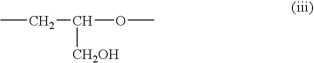
n represents the number of glycerol groups (iii) and is between 1 and 30 and preferably between 1 and 10.

Oxyalkylenated fatty alcohols that are particularly preferred according to the invention are saturated or unsaturated, linear or branched fatty alcohols containing from 10 to 20 carbon atoms and from 2 to 40 ethylene oxide groups.

As compounds of oxyalkylenated fatty alcohol type, mention may be made especially of the following marketed products:
Mergital LM2 (Cognis) [lauryl alcohol 2 EO];
Ifralan L12 (Ifrachem) and Rewopal 12 (Goldschmidt) [lauryl alcohol 12 EO];
Empilan KA 2.5/90FL (Albright & Wilson) and Mergital BL309 (Cognis) [decyl alcohol 3 EO];
Empilan KA 5/90 FL (Albright & Wilson) and Mergital BL589 (Cognis) [decyl alcohol 5 EO];
Brij 58 (Uniqema) and Simulsol 58 (SEPPIC) [cetyl alcohol 20 EO];
Emulgin 05 (Cognis) [oleocetyl alcohol 5 EO];
Mergital OC30 (Cognis) [oleocetyl alcohol 30 EO];
Brij 72 (Uniqema) [stearyl alcohol 2 EO];
Brij 76 (Uniqema) [stearyl alcohol 10 EO];
Brij 78P (Uniqema) [stearyl alcohol 20 EO];
Brij 700 (Uniqema) [stearyl alcohol 100 EO];
Emulgin BI (Cognis) [cetylstearyl alcohol 12 EO];
Emulgin L (Cognis) [cetyl alcohol 9 EO and 2 PO];
Witconol APM (Goldschmidt) [myristyl alcohol 3 PO].

As compounds of glycerolated fatty alcohol type, mention may be made especially of lauryl alcohol containing 4 mol of glycerol (INPCI name: Polyglyceryl-4 Lauryl Ether), oleyl alcohol containing 4 mol of glycerol (INPCI name: Polyglyceryl-4 Oleyl Ether), oleyl alcohol containing 2 mol of glycerol (INPCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The fatty alcohol may represent a mixture of fatty alcohols, which means that several fatty alcohol species may coexist in a commercial product, in the form of a mixture.

Advantageously, the fatty alcohol is solid or pasty at a temperature of 20° C. For the purposes of the present invention, the expression “fatty alcohol that is solid or pasty at 20° C.” means an alcohol that has a viscosity, measured with a rheometer at a shear rate of 1 s⁻¹, of greater than or equal to 1 Pa·s.

The fatty alcohol(s) may be present in the composition in a content of between 0.01% and 10%, preferably between 0.1% and 5% and more preferably between 0.2% and 4% by weight relative to the total weight of the composition.

For the purposes of the invention, the term “propellant” means any compound that is gaseous at a temperature of 20° C. and at atmospheric pressure, and that can be stored under pressure in liquid or gaseous form in an aerosol container.

The propellant may be chosen from optionally halogenated volatile hydrocarbons, such as n-butane, propane, isobutane, pentane or a halogenated hydrocarbon, and mixtures thereof. Carbon dioxide, nitrous oxide, dimethyl ether (DME), nitrogen or compressed air may also be used as propellant. Mixtures of propellants may also be used. Dimethyl ether and/or non-halogenated volatile hydrocarbons are preferably used.

The propellant(s) may be present in the composition in a content of between 1% and 15%, preferably between 2% and 10% and more preferably between 3% and 8% by weight relative to the total weight of the composition.

The composition according to the invention may also contain at least one additive chosen from fragrances, screening agents, nonionic, anionic, amphoteric or zwitterionic surfactants, preserving agents, proteins, vitamins, nonionic, anionic, cationic, amphoteric or zwitterionic polymers, mineral, plant or synthetic oils, non-amino silicones, plant waxes, ceramides, and any other additive conventionally used in cosmetic compositions, such as antidandruff agents, agents for preventing hair loss, dyes, pigments and reducing agents.

These additives are present in the composition in contents advantageously of between 0.001% and 20% by weight relative to the total weight of the composition. The precise amount of each additive depends on its nature and is readily determined by a person skilled in the art, and will depend on the selected hair application.

A person skilled in the art will take care to select the optional additives and the amount thereof such that they do not harm the properties of the compositions of the present invention.

These additives are present in the composition according to the invention in an amount ranging from 0 to 20% by weight relative to the total weight of the composition.

The cosmetic compositions for treating keratin materials in accordance with the invention are in the form of a foam and may be used in rinse-out or leave-in application. They are packaged in an aerosol device. This device generally comprises of a metal, glass or plastic container, optionally lined with a varnish, the said container being hermetically closed and provided with a withdrawal device allowing the product to exit in the form of a foam. The withdrawal device generally comprises a dip tube, a valve and a diffuser equipped with a nozzle.

The compositions in accordance with the invention may be used as haircare compositions, hair shaping compositions, shampoos, hair conditioners, hair conditioning compositions, such as compositions for giving the hair softness, or alternatively hair makeup compositions. The composition is preferably a shampoo or a hair conditioner.

A subject of the invention is also the use of the compositions according to the invention for the cosmetic treatment of the hair and the scalp.

Another subject of the invention is the use of the composition for washing and/or conditioning keratin materials such as the hair.

A subject of the present invention is also a cosmetic treatment process in which the cosmetic composition according to the invention is applied to wet or dry hair, this application optionally being followed by rinsing. The composition is advantageously applied after shampooing.

In one preferred embodiment of the invention, the compositions according to the invention are used as hair conditioners for treating the hair and the scalp.

In this case, they are applied to wet or dry hair, in amounts that are effective to treat it, this application being followed by rinsing.

The examples that follow are intended to illustrate the invention.

### EXAMPLE 1

A reference composition 1 (cream) and a composition 2 according to the invention (foam) were prepared.

| | Composition 1 | | Composition 2 | |
|---|---|---|---|---|
| Glyceryl stearate (1) | 1 | g | 0.95 | g |
| Dipalmitoylethylhydroxyethyl- | 4.5 | g | 4.27 | g |
| methylammonium methosulfate/ | | | | |
| cetearyl alcohol (30/70 by | | | | |
| weight) (2) | | | | |
| Cetyltrimethylammonium chloride | 3.2 | g | 3.04 | g |
| as an aqueous solution containing | | | | |
| 25% active material (3) | | | | |
| Polydimethylsiloxane containing | 0.6 | g | 0.57 | g |
| alpha-omega fatty-chain | | | | |
| quaternized ammonium acetate | | | | |
| groups, at 50% in propylene | | | | |
| glycol (4) | | | | |
| Isobutane/propane/butane mixture (5) | — | | 5 | g |
| Water | qs 100 | g | qs 100 | g |

(1) product sold by the company Goldschmidt
(2) product sold under the name Dehyquart F30 by the company Cognis
(3) product sold under the name Dehyquart AOR by the company Cognis
(4) product sold under the name Abil Quat 3272 by the company Goldschmidt
(5) product sold under the name Propel 45 by the company Repsol

Compositions 1 and 2 are applied to the hair, this application being followed by rinsing.

It is observed that the hair to which composition 2 according to the invention is applied is lighter, more individualized, easier to disentangle and smoother than the hair to which composition 1 is applied.

### EXAMPLE 2

A reference composition 1 (cream) and a composition 2 according to the invention (foam) were prepared.

| | Composition 1 | | Composition 2 | |
|---|---|---|---|---|
| Cetylstearyl alcohol (30/70 | 0.5 | g | 0.47 | g |
| C16/C18) (6) | | | | |
| Glyceryl stearate (1) | 1 | g | 0.95 | g |
| Dipalmitoylethylhydroxyethyl- | 4.5 | g | 4.27 | g |
| methylammonium methosulfate/ | | | | |
| cetearyl alcohol (2) | | | | |
| Cetyltrimethylammonium chloride | 3.2 | g | 3.04 | g |
| as an aqueous solution (3) | | | | |
| Polydimethylsiloxane containing | 0.75 | g | 0.71 | g |
| alpha-omega fatty-chain | | | | |
| quaternized ammonium acetate | | | | |
| groups, at 50% in propylene glycol (4) | | | | |
| Polydimethylsiloxane containing | 0.75 | g | 0.71 | g |
| alpha-omega fatty-coco-chain | | | | |
| quaternized ammonium acetate | | | | |
| groups, as a 95% solution in | | | | |
| propylene glycol (7) | | | | |
| Isobutane/propane/butane mixture | — | | 5 | g |
| Water | qs 100 | g | qs 100 | g |

(6) product sold under the name Crodacol 1618 by the company Croda
(7) product sold under the name Abil Quat 3474 by the company Goldschmidt

Compositions 1 and 2 are applied to the hair, this application being followed by rinsing. The application of the foam is easier than that of the cream and affords better distribution from the root to the end.

It is observed that the hair to which composition 2 according to the invention is applied is lighter, more individualized, easier to disentangle and smoother than the hair to which composition 1 is applied.

### EXAMPLE 3

A composition according to the invention (foam) was prepared.

| | Composition | |
|---|---|---|
| Cetylstearyl alcohol (30/70 C16/C18) (6) | 2.8 | g |
| Palm oil | 1 | g |
| Cetyltrimethylammonium chloride as an aqueous | 3.04 | g |
| solution (3) | | |
| Polydimethylsiloxane containing alpha-omega | 0.57 | g |
| fatty-chain quaternized ammonium acetate | | |
| groups, at 50% in propylene glycol (4) | | |
| Isobutane/propane/butane mixture | 5 | g |
| Water | qs 100 | g |

The composition is applied to the hair, this application being followed by rinsing.

It is observed that the hair is light, well individualized and easy to disentangle and to smooth out.

As used herein, the phrases “selected from the group consisting of,” “chosen from,” “selected from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

We claim:

1. A composition comprising, in a cosmetically acceptable medium, at least one cationic surfactant, at least one silicone chosen from amino silicones and vinyl silicones, at least one fatty alcohol, and at least one propellant.

2. The composition according to claim 1, wherein the cationic surfactant is chosen from salts of optionally polyoxyalkylenated primary, secondary or tertiary fatty amines; quaternary ammonium salts.

3. The composition according to claim 1, comprising at least one quaternary ammonium salts chosen from:
those of general formula (V) below: 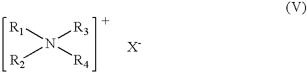
in which the symbols R₁ to R₄, which may be identical or different, represent a linear or branched aliphatic radical containing from 1 to 30 carbon atoms, or an aromatic radical such as aryl or alkylaryl; X⁻ is an anion chosen from the group of halides, phosphates, acetates, lactates, (C₂-C₆) alkyl sulfates and alkyl- or alkylarylsulfonates;
the quaternary ammonium salts of imidazoline of formula (VI) below: 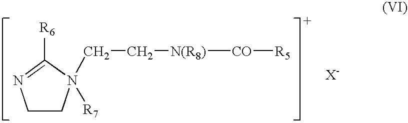
in which R₅ represents an alkenyl or alkyl radical containing from 8 to 30 carbon atoms, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X is an anion chosen from the group of halides, phosphates, acetates, lactates, alkyl sulfates, alkyl sulfonates or alkylaryl sulfonates;
the diquaternary ammonium salts of formula (VII): 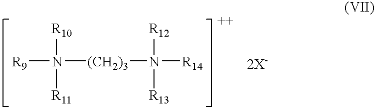
in which R₉ denotes an aliphatic radical containing from about 16 to 30 carbon atoms, R₁₀, R₁₁, R₁₂, R₁₃ and R₁₄, which may be identical or different, are chosen from hydrogen and an alkyl radical containing from 1 to 4 carbon atoms, and X⁻ is an anion chosen from the group of halides, acetates, phosphates, nitrates, ethyl sulfates and methyl sulfates;
the quaternary ammonium salts containing at least one ester function, of formula (VIII) below: 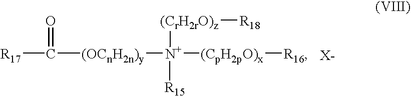
in which:
R₁₅ is chosen from C₁-C₆ alkyl radicals and C₁-C₆ hydroxyalkyl or dihydroxyalkyl radicals;
R₁₆ is chosen from:
a radical 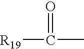
linear or branched, saturated or unsaturated C₁-C₂₂ hydrocarbon-based radicals R₂₀,
a hydrogen atom,
R₁₇ is chosen from:
a radical 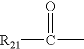
linear or branched, saturated or unsaturated C₁-C₆ hydrocarbon-based radicals R₂₂,
a hydrogen atom,
R₁₇, R₁₉ and R₂₁, which may be identical or different, are chosen from linear or branched, saturated or unsaturated C₇-C₂, hydrocarbon-based radicals;
r, n and p, which may be identical or different, are integers ranging from 2 to 6;
y is an integer ranging from 1 to 10;
x and z, which may be identical or different, are integers ranging from 0 to 10;
X⁻ is a simple or complex organic or inorganic anion;
with the proviso that the sum x+y+z is from 1 to 15, that when x is 0, then R₁₆ denotes R₂₀ and that when z is 0, then R₁₈ denotes R₂₂.

4. The composition according to claim 3, wherein the compound of formula (VIII) is chosen from the salts of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof.

5. The composition according to claim 3, wherein the surfactant of formula (V) is chosen from dipalmitoylethylhydroxy-ethylmethylammonium salts, behenyltrimethylammonium, distearyldimethylammonium, cetyltrimethylammonium and benzyldimethylstearylammonium salts, palmitylamidopropyltrimethylammonium salts and stearamidopropyldimethyl(myristyl acetate)ammonium salts.

6. The composition according to claim 1, wherein the cationic surfactant is chosen from behenyltrimethylammonium chloride, cetyltrimethylammonium chloride, quaternium-83, behenylamidopropyl 2,3-dihydroxypropyl dimethyl ammonium chloride and palmitoylamidopropyltrimethylammonium chloride.

7. The composition according to claim 1, wherein the cationic surfactant is present in the composition in a content of between 0.01% and 10% by weight relative to the total weight of the composition.

8. The composition according to claim 1, wherein the amino silicone is chosen from:
(a) the polysiloxanes referred to in the CTFA dictionary as “amodimethicone” and corresponding to the formula: 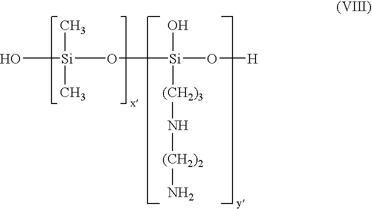
in which x′ and y′ are integers such that the said number-average molecular weight is between 5000 and 500 000;
(b) amino silicones corresponding to the formula:
R′ₐG₃₋ₐ-Si(OSiG₂)ₙ—(OSiG_{b}R′_{2-b})ₘ—O—SiG₃₋ₐ—R′ₐ (IX)
in which:
G is a hydrogen atom or a phenyl, OH or C₁-C₈ alkyl group,
a denotes the number 0 or an integer from 1 to 3,
b denotes 0 or 1,
m and n are numbers such that the sum (n+m) can range especially from 1 to 2000 and in particular from 50 to 150, n possibly denoting a number from 0 to 1999 and especially from 49 to 149 and m possibly denoting a number from 1 to 2000, and especially from 1 to 10;
R′ is a monovalent radical of formula —C_{q}H_{2q}L in which q is a number from 2 to 8 and L is an optionally quaternized amine group chosen from the groups:
—NR″-Q-N′(R″)2
—N(R″)2
—N⊕(R″)3 A-
—NH⊕(R″)2 A-
—NH2⊕(R″) A-
—N(R″)-Q-N⊕R″H2 A-
—NR″-Q-N⊕(R″)2H A-
—NR″-Q-N⊕(R″)₃ A-,
in which R″ can denote hydrogen, phenyl, benzyl or a saturated monovalent hydrocarbon-based radical; Q denotes a linear or branched group of formula CᵣH₂ᵣ, r being a n integer ranging from 2 to 6 and preferably from 2 to 4; and A⁻ represents a halide ion,
(c) amino silicones corresponding to the formula: 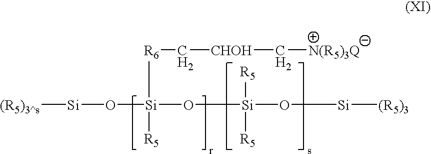
in which:
R₅ represents a monovalent hydrocarbon-based radical containing from 1 to 18 carbon atoms, and in particular a C₁-C₁₈ alkyl or C₂-C₁₈ alkenyl radical;
R₆ represents a divalent hydrocarbon-based radical, in particular a C₁-C₁₈ alkylene radical or a divalent C₁-C₁₈ alkylenoxy radical;
Q⁻ is an anion such as a halide ion or an organic acid salt;
r represents an average statistical value from 2 to 20 and in particular from 2 to 8;
s represents an average statistical value from 20 to 200 and in particular from 20 to 50,
d) quaternary ammonium silicones of formula: 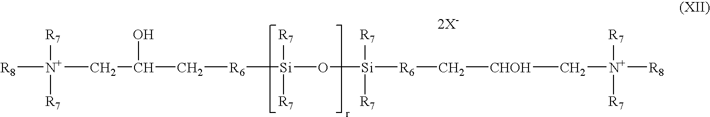
in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms;
R₆ represents a divalent hydrocarbon-based radical, in particular a C₁-C₁₈ alkylene radical or a divalent C₁-C₁₈ alkylenoxy radical;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide ion or an organic acid salt;
r represents an average statistical value from 2 to 200 and in particular from 5 to 100;
e) amino silicones of formula (XIII): 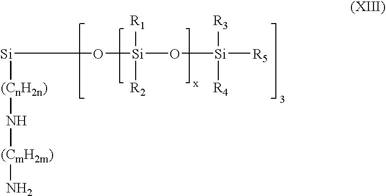
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5,
and in which x is chosen such that the amine number is between 0.01 and 1 meq/g.

9. The composition according to claim 8, wherein the amino silicone of formula (IX) is the silicone known as trimethylsilyl amodimethicone, corresponding to the formula: 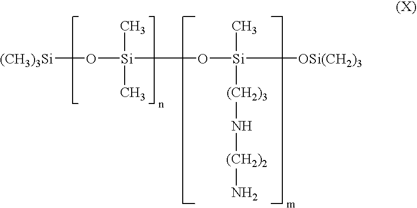
in which n and m have the same meanings as in formula (IX).

10. Composition according to claim 1, wherein the amino silicone is chosen from amodimethicones and trimethylsilyl amodimethicones, and silicones containing quaternary ammonium groups.

11. The composition according to claim 1, wherein the vinyl silicone is chosen from dimethylsiloxane copolymers containing α,ω-divinyl/α,ω-dihydrogenopolydimethylsiloxane groups.

12. The composition according to claim 1, wherein the silicone chosen from amino silicones and vinyl silicones is present in the composition in a content of between 0.01% and 6% by weight relative to the total weight of the composition.

13. The composition according to claim 1, wherein the fatty alcohol is linear or branched, and saturated or unsaturated.

14. The composition according to claim 13, wherein the fatty alcohol is chosen from fatty alcohols containing from 10 to 20 carbon atoms and 2 to 40 ethylene oxide groups.

15. The composition according to claim 13, wherein the fatty alcohol has the structure R—OH, in which R denotes a saturated or unsaturated, linear or branched radical containing from 8 to 40 carbon atoms.

16. The composition according to claim 13, wherein the fatty alcohol is chosen from cetyl alcohol, stearyl alcohol and oleyl alcohol, and mixtures thereof.

17. The composition according to claim 13, wherein the fatty alcohol is chosen from lauryl alcohol containing 4 mol of glycerol, oleyl alcohol containing 4 mol of glycerol, oleyl alcohol containing 2 mol of glycerol, cetearyl alcohol containing 2 mol of glycerol, cetearyl alcohol containing 6 mol of glycerol, oleocetyl alcohol containing 6 mol of glycerol, and octadecanol containing 6 mol of glycerol.

18. The composition according to claim 1, wherein the fatty alcohol is solid or pasty at room temperature.

19. The composition according to claim 1, wherein the fatty alcohol is present in the composition in a content of between 0.01% and 10% by weight relative to the total weight of the composition.

20. The composition according to claim 1, wherein the propellant is chosen from optionally halogenated volatile hydrocarbons, carbon dioxide, nitrous oxide, dimethyl ether (DME), nitrogen and compressed air, and mixtures thereof.

21. The composition according to claim 20, wherein the volatile hydrocarbon is chosen from n-butane, propane, isobutane and pentane, and mixtures thereof.

22. The composition according to claim 20, wherein the propellant is dimethyl ether.

23. The composition according to claim 1, wherein the propellant is present in the composition in a content of between 1% and 15% by weight relative to the total weight of the composition.

24. The composition according to claim 1, wherein the cosmetically acceptable medium comprises of water or of one or more cosmetically acceptable solvents, or of water-solvent mixtures.

25. The composition according to claim 24, wherein the cosmetically acceptable solvent is chosen from ethanol and isopropanol.

26. The composition according to claim 25, wherein the cosmetically acceptable solvent is ethanol.

27. The composition according to claim 1, wherein it also contains at least one additive chosen from fragrances, screening agents, nonionic, anionic, amphoteric or zwitterionic surfactants, preserving agents, proteins, vitamins, nonionic, anionic, cationic, amphoteric or zwitterionic polymers, mineral, plant or synthetic oils, antidandruff agents, agents for preventing hair loss, dyes, pigments, reducing agents, non-amino silicones, plant waxes, and ceramides.

28. An aerosol device comprising a composition according to claim 1.

29. A method, comprising applying the composition of claim 1 to the hair and/or the scalp.

30. The method according to claim 29, wherein said composition is applied to the hair.

31. The method according to claim 30, wherein the composition is applied after shampooing.
